(19) United States
(12) Patent Application Publication
(10) Pub. No.: US 2023/0355943 A1
(43) Pub. Date: Nov. 9, 2023

(54) Full Featured Integrated Midline Catheter System with Stabilization and Near Patient Access Port Blood Draw, Infusion, and Digital Probes

(71) Applicant: Becton, Dickinson and Company, Franklin Lakes, NJ (US)

(72) Inventor: Jonathan Karl Burkholz, Salt Lake City, UT (US)

(21) Appl. No.: 18/143,318

(22) Filed: May 4, 2023

Related U.S. Application Data
(60) Provisional application No. 63/338,684, filed on May 5, 2022.

(51) Int. Cl. A61M 39/02; A61M 25/09; A61M 25/06

(52) U.S. Cl. CPC A61M 39/0247; A61M 25/09; A61M 25/0637; A61M 25/0606; A61M 2039/0258; A61M 2039/027; A61M 2039/0202; A61M 2039/0205

(57) ABSTRACT

An integrated midline catheter system including a catheter adapter having a catheter extending therefrom, a stabilization platform, a proximal connector portion fluidly coupled to the catheter, and a side branch fluidly coupled to the catheter. The system also includes a near patient access port coupled to the side branch of the catheter adapter, wherein the near patient access port includes a connector portion couplable to a peripheral probe device. Furthermore, the system includes a catheter insertion device, wherein the catheter insertion device is configured to enable distal movement of the catheter adapter and catheter so as to position the catheter within a patient's vasculature.

### CROSS-REFERENCE TO RELATED APPLICATION

The present application claims priority to U.S. Provisional Application Ser. No. 63/338,684, entitled “Full Featured Integrated Midline Catheter System with Stabilization and Near Patient Access Port Blood Draw, Infusion, and Digital Probes”, filed May 5, 2022, the entire disclosure of which is hereby incorporated by reference.

### BACKGROUND OF THE INVENTION

### Field of the Invention

The present disclosure generally relates to integrated midline catheter systems. More particularly, the integrated midline catheter systems may include catheter adapter stabilization and a near patient access port configured for blood draw, infusion, and/or insertion of digital probes into the patient's vasculature via the indwelling midline catheter.

### Description of Related Art

Midline catheters are generally used for parenteral nutrition, IV fluid replacement, and/or the administration of analgesics and antibiotics. Midline catheters are inserted at the bedside using sterile techniques and can remain in place for several weeks. The insertion (venipuncture) is performed above and below the antecubital fossa in the cephalic, basilica, or bronchial veins. The catheter may be of various lengths and gauges, with the tip of the catheter terminating below the axilla and proximal central veins.

The potential advantages of a midline catheter are the reduced frequency of repeated venipunctures for labs/restarts, decreased incidence of catheter related infections, extended implant/indwell duration, improved clinical outcomes, patient satisfaction and associated cost savings. Placing the catheter tip in the larger diameter veins of the upper arm compared to smaller veins provides for improvements in drug delivery therapy and hemodilution. Midline catheters can also be used for infusing contrast media at higher flow rates that are typically done by other catheters such as, e.g., peripheral intravenous catheters (PIVCs).

However, an advantage of vascular access devices such as PIVCs over midline catheters is the recent development of systems to aid in blood draw and/or in-vein digital measurements directly from an indwelling catheter of the vascular access device. For example, a blood draw device known as PIVO™ from Becton, Dickinson and Company, is configured as a single-use device which temporarily attaches to a PIVC to draw a blood sample. Using an existing peripheral intravenous line as a conduit to the vasculature, the PIVO™ device advances a flexible, internal probe or flow tube through the PIVC to (or beyond) the catheter tip to collect a blood sample. This flow tube is designed to extend beyond the suboptimal draw conditions around the indwelling line to reach vein locations where blood flow is optimal for aspiration. Once blood collection is complete, the flow tube is retracted, and the device is removed from the PIVC and discarded. An example of one such blood draw device is shown and described in U.S. Pat. No. 10,300,247 B2, which is incorporated by reference herein in its entirety. Similarly, devices configured direct probes, wires, fibers, guidewires, sensors, etc. through the PIVC and into the patient's vasculature for in-vein digital measurements have also been developed. On the other hand, conventional midline catheter systems lack the access architecture needed for compatibility with such blood draw and/or in-vein digital measurement devices.

### SUMMARY OF THE INVENTION

Accordingly, the present disclosure generally relates to an integrated midline catheter system. In accordance with an aspect of the present disclosure, the system includes a catheter adapter having a catheter extending therefrom, a stabilization platform, a proximal connector portion fluidly coupled to the catheter, and a side branch fluidly coupled to the catheter. The system also includes a near patient access port coupled to the side branch of the catheter adapter, wherein the near patient access port includes a connector portion couplable to a peripheral probe device. The system further includes a catheter insertion device, wherein the catheter insertion device is configured to enable distal movement of the catheter adapter and catheter so as to position the catheter within a patient's vasculature.

In some embodiments, the near patient access port further includes a secondary port, and the secondary port is coupled to an integrated extension set.

In some embodiments, the system further includes a proximal access port coupled to a proximal end portion of the integrated extension set.

In some embodiments, the proximal access port is color-coded to provide a flow rate indication.

In some embodiments, the proximal access port comprises indicia indicative of at least one of catheter length and catheter gauge.

In some embodiments, the connector portion of the near patient access port is a needle-free connector.

In some embodiments, the needle-free connector is a split septum needle-free connector.

In some embodiments, the near patient access port further includes a high-pressure injection port.

In some embodiments, the high-pressure injection port is color-coded to provide a flow rate indication.

In some embodiments, the high-pressure injection port includes indicia indicative of at least one of catheter length and catheter gauge.

In some embodiments, the stabilization platform includes a pair of stabilizing wings.

In some embodiments, each of the stabilizing wings includes a gripping surface to provide a grip for the distal movement of the catheter adapter and catheter along the catheter insertion device.

In some embodiments, the system includes a pair of catheter advancement tabs extending from the catheter adapter and configured to provide to provide a grip for the distal movement of the catheter adapter and catheter along the catheter insertion device.

In some embodiments, the catheter advancement tabs are detachable from the catheter adapter.

In some embodiments, the catheter insertion device further includes a housing, an introducer needle extending distally from the housing, and an actuator tab slidable along the housing to selectively advance a guidewire through the introducer needle, wherein the catheter is configured to selectively advance over the introducer needle and the guidewire.

In some embodiments, the catheter insertion device is detachable from the catheter adapter after insertion of the catheter within the patient's vasculature.

According to another aspect of the present disclosure, an integrated midline catheter system is disclosed. The catheter system includes a catheter adapter having a catheter extending therefrom, a stabilization platform, a proximal connector portion fluidly coupled to the catheter, and a side branch fluidly coupled to the catheter. The system also includes a near patient access port coupled to the side branch of the catheter adapter, wherein the near patient access port comprises a connector portion couplable to a peripheral probe device.

In some embodiments, the catheter is between 8 cm and 10 cm in length.

In some embodiments, the near patient access port further includes a secondary port, and the secondary port is coupled to an integrated extension set.

In some embodiments, the connector portion of the near patient access port is a needle-free connector.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is an isometric view of an integrated midline catheter system with a catheter insertion device, stabilization, and a near patient access port in accordance with an aspect of the present disclosure;

FIG. 2 is an isometric view of the integrated midline catheter, stabilization, and near patient access port of FIG. 1;

FIG. 3 is an isometric view of an integrated midline catheter system with a catheter insertion device, stabilization, and a near patient access port in accordance with another aspect of the present disclosure;

FIG. 4 is an isometric view of an integrated midline catheter system with a catheter insertion device, stabilization, and a near patient access port in accordance with another aspect of the present disclosure; and

FIG. 5 is an isometric view of an integrated midline catheter system with a catheter insertion device, stabilization, and a near patient access port in accordance with another aspect of the present disclosure.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward distal tip of the needle or catheter of the system, and the terms “in the proximal direction” and “proximally” mean in the direction opposite the direction of the distal tip of the needle or catheter.

Embodiments of the present disclosure will primarily be described in the context of devices for use with integrated midline catheters. However, embodiments of the present disclosure equally extend to use with other catheter devices.

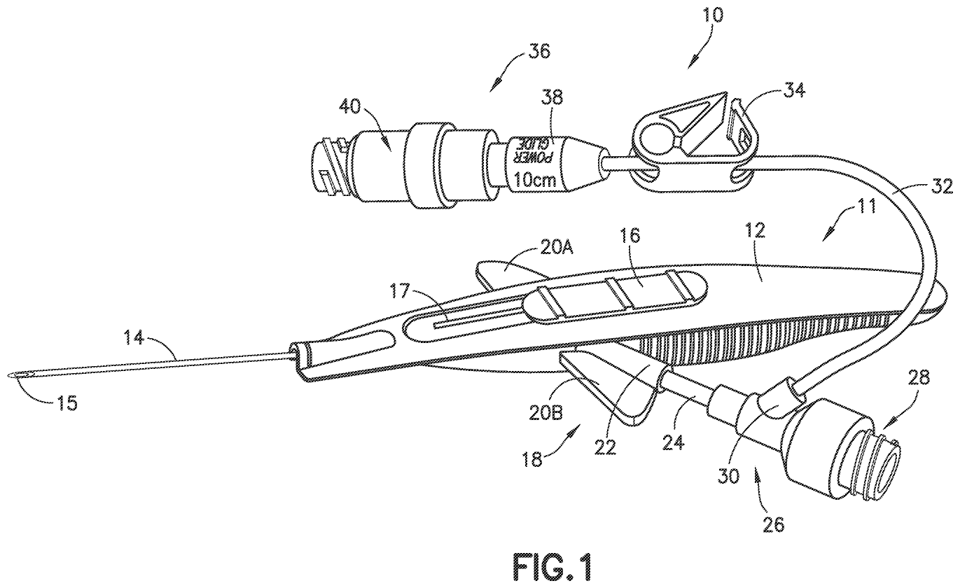

Referring to FIG. 1, an integrated midline catheter system 10 in accordance with an aspect of the present disclosure is illustrated. In a first configuration, the system 10 includes a catheter insertion device 11 having a housing 12. An introducer needle 14 extends from a distal end of the housing 12, with the introducer needle 14 having a distal tip 15. Due to the length of the introducer needle 14, a distal end portion of the housing 12 may include a feature or surface to at least partially support an intermediate portion of the introducer needle 14 during insertion, thereby reducing the flexibility of the introducer needle 14 during insertion.

An actuator tab 16 is positioned on a surface of the housing 12, with the actuator tab 16 configured for substantially linear movement along a slot 17 formed in the housing 12. While not shown in FIG. 1, the actuator tab 16 may be operably coupled to a guidewire, with the guidewire being sized and configured to selectively pass through (and beyond) the introducer needle 14 as actuator tab 16 is advanced distally along the slot 17 and, conversely, to retract through the introducer needle 14 when the actuator tab 16 is retracted proximally by the clinician. The guidewire may include a blunt, atraumatic tip to substantially prevent vascular damage. In some embodiments, a polymer rod or tubing with an atraumatic tip may replace the guidewire.

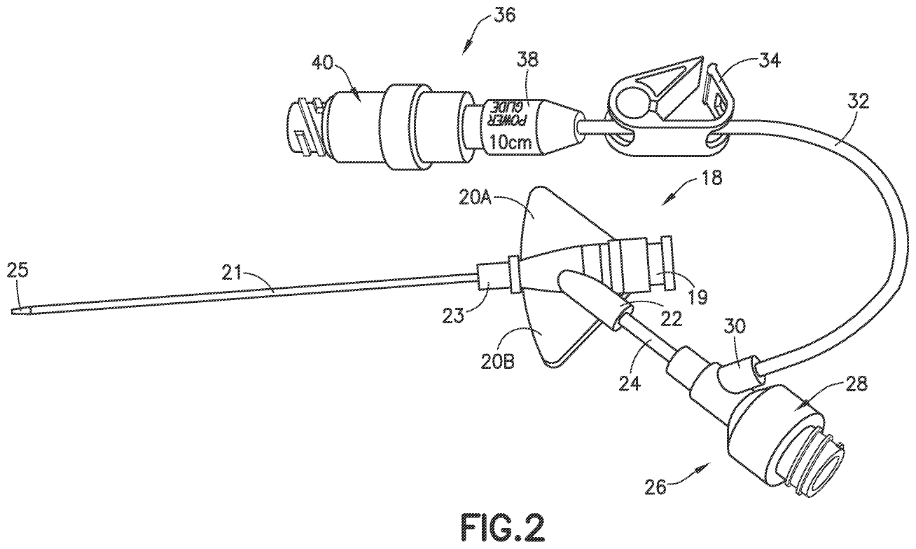

Still referring to FIG. 1, and also to FIG. 2, system 10 further includes a catheter adapter 18 having a stabilization platform formed of opposing stabilizing wings 20A, 20B. In the first configuration shown in FIG. 1, the catheter adapter 18 is disposed at least partially within the housing 12 such that the stabilizing wings 20A, 20B extend externally from opposing lateral sides of the housing 12. The catheter adapter 18 is configured to move linearly along the housing 12 in a distal direction such that a catheter 21 (shown in FIG. 2) may be selectively advanced over the introducer needle 14 and into the patient's vasculature. The catheter 21 may be any appropriate length and gauge for use as a midline catheter. For example, catheter 21 may have a length between 8 cm and 10 cm, and may be one of 16 GA, 18 GA, 20 GA, or 22 GA. However, it is to be understood that catheter 21 is not limited to the above-referenced lengths and/or gauges. Additionally, the catheter 21 may be formed of any appropriate material such as, e.g., polyurethane. The catheter 21 may have a reinforced tip 25, which may be configured to substantially prevent the catheter tip from collapsing and/or aid in deployment and positioning of the catheter 21.

Placement of the catheter 21 into the patient's vasculature utilizing the system 10 will now be explained in accordance with an embodiment of the present disclosure. First, the clinician identifies the appropriate insertion site, and cleans and prepares the insertion site in accordance with institutional policy. The clinician then grips and advances the entire catheter insertion device 11 to insert the introducer needle 14 into an appropriate vein or other vascular access location of the patient. While not shown, the system may include magnetic needle guidance to ensure proper insertion of the introducer needle 14. The magnetic needle guidance may be used in conjunction with ultrasound placement system and methods such as, e.g., the Cue Needle Tracking System from Becton, Dickinson and Co. With the introducer needle 14 in place, the clinician may then advance the actuator tab 16 distally along the slot 17 such that a guidewire (now shown) operably coupled to the actuator tab 16 simultaneously advances through the introducer needle 14, thereby providing an extended guided path for deployment of the catheter 21.

Next, with the guidewire in an advanced position, the clinician may distally advance the catheter adapter 18 along the housing 12, thereby also advancing the catheter 21 over the introducer needle 14 and guidewire, moving the catheter 21 into a desired position within the patient's vasculature. To distally advance the catheter adapter 18, the clinician may grip or otherwise manipulate one or both of the stabilizing wings 20A, 20B. Additionally and/or alternatively, the clinician may utilize a side branch 22 extending from the catheter adapter 18 to aid in advancement of the catheter adapter 18 and catheter 21.

With the catheter 21 and catheter adapter 18 in a desired position, the clinician may separate the catheter insertion device 11 from the catheter adapter 18. In some embodiments, the housing 12 of the catheter insertion device 11 may at least partially split after placement of the catheter 21, allowing the housing 12 separate from the catheter adapter 18. In doing so, the introducer needle 14 and guidewire (not shown) are also withdrawn from the catheter 21 and are pulled through a self-sealing proximal connector portion 19 of the catheter adapter 18. Accordingly, with the catheter insertion device 11 removed, the catheter adapter 18 remains in place at the insertion site in the second configuration shown in FIG. 2, with the stabilizing wings 20A, 20B configured to stabilize the catheter adapter 18. In some embodiments, a strain relief feature 23 may be provided between the catheter adapter 18 and the catheter 21.

Referring still to FIGS. 1 and 2, the system 10 further includes a near patient access port 26. Near patient access port 26 is configured to provide catheter access to peripheral devices such as, e.g., a blood draw device (e.g., PIVO™ from Becton, Dickinson and Company), or a vascular access probe (VAP) for in-vein digital measurement of patient date such as temperature, pH, lactate, and/or other blood-based measurements. In some embodiments, the near patient access port 26 is coupled to the side branch 22 of catheter adapter 18 via a length of intermediate tubing 24. However, in other embodiments, it is to be understood that near patient access port 26 may be coupled directly to the side branch 22, or connected via another intermediate member.

Near patient access port 26 includes a connector portion 28. In some embodiments, connector portion 28 is configured to be compatible with peripheral devices such as blood draw devices and/or vascular access probes. The connector portion 28 may include an interface for secure coupling of the peripheral device(s) to the connector portion 28. In some embodiments, the connector portion 28 is configured as a needle-free connector (NFC) configured to receive, e.g., a blunt introducer of a blood draw device. More specifically, the connector portion 28 may be configured as a split-septum NFC with direct probe access such as, e.g., Q-Syte™ or SmartSite™ NFCs from Becton, Dickinson and Co., or any other appropriate split-septum NFC. Alternatively, in other embodiments, the connector portion 28 may be formed of a non-split-septum-type NFC. Furthermore, in some embodiments, the near patient access port 26 may include anti-microbial and/or flush-promoting features. For example, the near patient access port 26 may include one or more of an offset tubing port vortex-creating feature, a proximal flow-diverting feature, anti-microbial NFC lubricant, anti-microbial eluting surface coating(s) or insert(s), etc.

With near patient access port 26 fluidly coupled to the catheter adapter 18 via the side port 22, the system 10 provides for probe (or tube) access from a peripheral probe device through the indwelling catheter 21. As catheter 21 is a midline catheter and is generally longer in length than, e.g., a PIVC, the length probe or tube of the peripheral probe device may be altered and/or optimized for use with the midline catheter, thereby enabling the probe or tube to potentially extend beyond the reinforced tip 25 of the catheter 21 when deployed.

Near patient access port 26 further includes a secondary port 30 positioned near a distal end thereof. In some embodiments, the secondary port 30 is coupled to an integrated extension set 32, with the integrated extension set 32 further being coupled to a proximal portion 36 at a proximal end thereof. A clamp 34 may be provided on the integrated extension set 32, with the clamp 34 configured to selectively restrict flow through the integrated extension set 32. In some embodiments, the clamp 34 may be color-coded to signify the type and/or injection compatibility of the integrated extension set 32.

In some embodiments, the proximal portion 36 may include a proximal access port 38 and a proximal connector 40. The proximal connector 40 may be removably or non-removably coupled to the proximal access port 38, and proximal connector 40 may be configured to allow fluid infusion through the catheter 21 via the near patient access port 26. In some embodiments, the proximal access port 38 may be color-coded and/or may contain indicia to indicate catheter length, catheter gauge, high-pressure injection compatibility, etc. In other embodiments, during insertion of the catheter 21 into the patient's vasculature, the proximal connector 40 may be replaced with, e.g., a removable vent plug. In some embodiments, the proximal access port 38 may be a non-split-septum connector, and may include antimicrobial and/or flush-ability features. While only a single proximal access port 38 is shown, it is to be understood that the proximal portion 36 may be configured to include more than one access port.

In the embodiments shown in FIGS. 1 and 2, the secondary port 30 of the near patient access port 26 is configured as an angled port, resulting in the near patient access port 26 being a y-shaped adapter. However, in other embodiments, the secondary port 30 may be configured as a t-shaped adapter such that the secondary port 30 enters the near patient access port 26 at a substantially 90° angle. Additionally, while secondary port 30 is shown as being directed towards the center of the device, it is to be understood that secondary port 30 may be directed away from the device.

Accordingly, the integrated midline catheter system 10 described above with respect to FIGS. 1 and 2 provides a midline catheter system with a near patient access port compatible with instrument, probe, and/or tubing delivery through the midline catheter and into the patient's vascular system, which conventional integrated midline catheter systems do not provide.

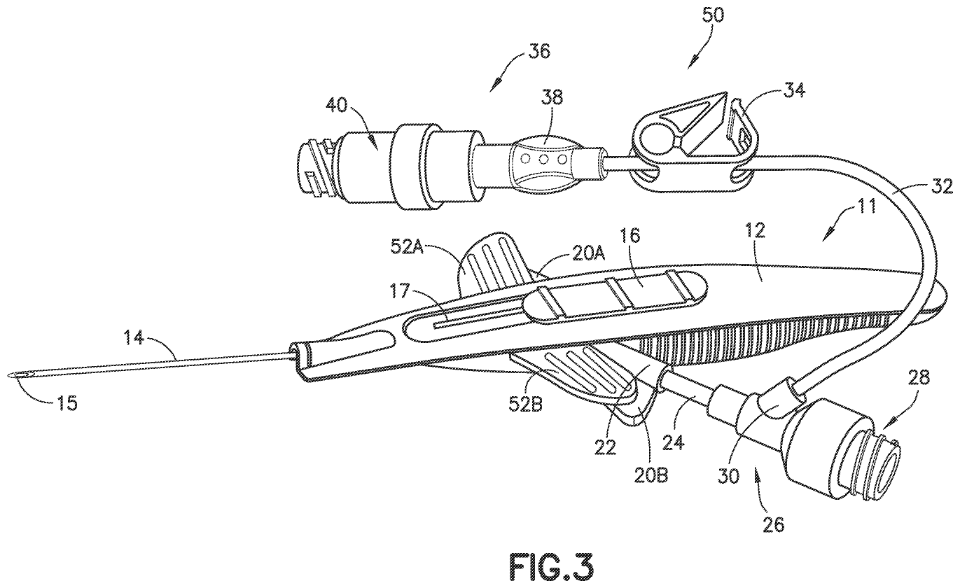

Referring now to FIG. 3, an integrated midline catheter system 50 in accordance with another aspect of the present disclosure is shown. System 50 includes many similar features as system 10 described above with respect to FIGS. 1 and 2 and, as such, like reference numerals are used throughout. However, unlike system 10, which utilized only stabilizing wings 20A, 20B and/or side branch 22 of the catheter adapter 18 to advance the catheter adapter 18 and catheter 21 distally relative to housing 12, the catheter insertion device 51 as shown in FIG. 3 includes a pair of catheter advancement tabs 52A, 52B extending from opposing sides of the housing 12, with the catheter advancement tabs 52A, 52B being separate from the stabilizing wings 20A, 20B of the stabilization platform.

The catheter advancement tabs 52A, 52B are operably coupled to the catheter adapter 18 (not shown in FIG. 3) so as to provide grips for the clinician to advance the catheter adapter 18 and catheter 21 distally along the housing 12. In some embodiments, when the clinician retracts and decouples the catheter insertion device 51 after the catheter is in place within the patient's vasculature, the catheter advancement tabs 52A, 52B remain in place with the catheter adapter 18. In some embodiments, the catheter advancement tabs 52A, 52B may be separable and removable from the catheter adapter 18 after placement of the catheter. Additionally and/or alternatively, in some embodiments, the catheter advancement tabs 52A, 52B may be color-coded according to the length and/or gauge of the catheter housed within the catheter insertion device 51. Furthermore, while two catheter advancement tabs 52A, 52B are shown in FIG. 3, it is to be understood that only one catheter advancement tab may be provided.

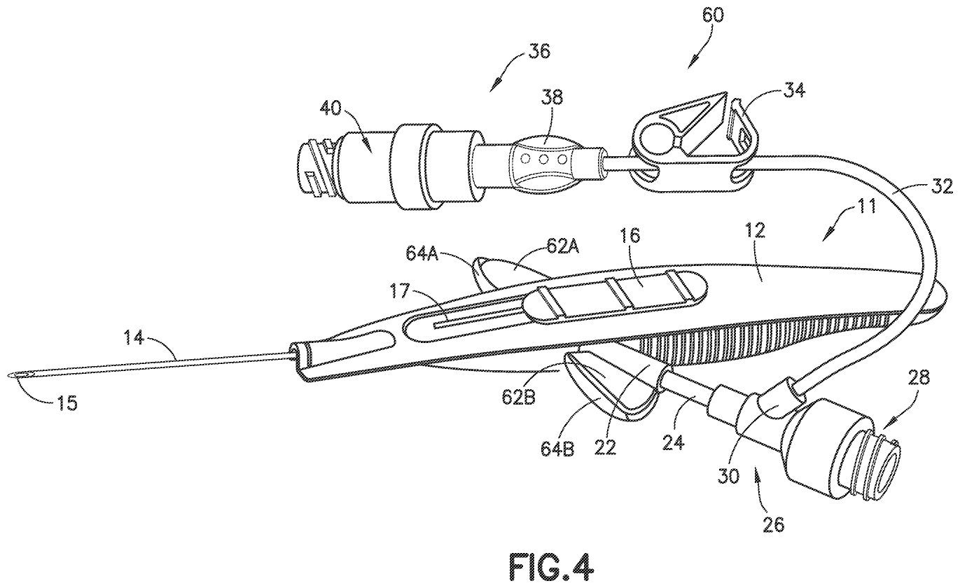

Next, referring to FIG. 4, an integrated midline catheter system 60 in accordance with another aspect of the present disclosure is shown. Again, system 60 includes many similar features as system 10 described above with respect to FIGS. 1 and 2 and, as such, like reference numerals are used throughout. However, unlike system 50, which utilized separate catheter advancement tabs 52A, 52B advance the catheter adapter 18 and catheter 21 (not shown in FIG. 4) distally relative to housing 12, the system 60 as shown in FIG. 4 includes a pair of stabilizing wings 62A, 62B extending from opposing sides of the housing 12 to form a stabilizing platform for the catheter adapter, with the stabilizing wings 62A, 62B incorporating respective gripping surfaces 64A, 64B thereon. One or both of the gripping surfaces 64A, 64B may be gripped by the clinician to advance the catheter adapter 18 and catheter 21 distally along the housing 12.

In some embodiments, when the clinician retracts and decouples the catheter insertion device 61 after the catheter is in place within the patient's vasculature, and the stabilizing wings 62A, 62B with gripping surfaces 64A, 64B remain in place with the catheter adapter 18. While gripping surfaces 64A, 64B are shown as upwardly curved surfaces on the stabilizing wings 62A, 62B, it is to be understood that other shapes and/or configurations of gripping surfaces may be utilized on the stabilizing wings 62A, 62B. Furthermore, while two gripping surfaces 64A, 64B are shown in FIG. 4, it is to be understood that only one gripping surface may be provided.

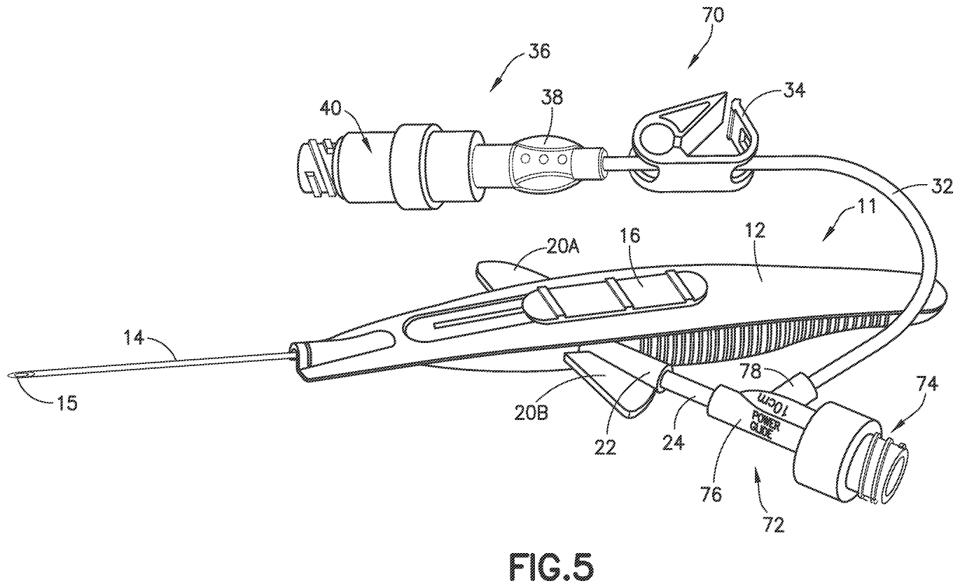

Referring now to FIG. 5, an integrated midline catheter system 70 in accordance with another aspect of the present disclosure is shown. As with systems 50 and 60 described above, system 70 includes many similar features as system 10 described above with respect to FIGS. 1 and 2 and, as such, like reference numerals are used throughout. However, system 70 includes a near patient access port 72 configured for high-pressure injection. Additionally, near patient access port 72 is configured to provide catheter access to peripheral devices such as, e.g., a blood draw device (e.g., PIVO™ from Becton, Dickinson and Company), or a vascular access probe (VAP) for in-vein digital measurement of patient date such as temperature, pH, lactate, and/or other blood-based measurements.

Near patient access port 72 includes a connector portion 74. In some embodiments, connector portion 74 is configured to be compatible with peripheral devices such as blood draw devices and/or vascular access probes. The connector portion 74 may include an interface for secure coupling of the peripheral device(s) to the connector portion 74. In some embodiments, the connector portion 74 is configured as a needle-free connector (NFC) configured to receive, e.g., a blunt introducer of a blood draw device. More specifically, the connector portion 74 may be configured as a split-septum NFC with direct probe access such as, e.g., Q-Syte™ or SmartSite™ NFCs from Becton, Dickinson and Co., or any other appropriate split-septum NFC. Alternatively, in other embodiments, the connector portion 74 may be formed of a non-split-septum-type NFC. Furthermore, in some embodiments, the near patient access port 72 may include anti-microbial and/or flush-promoting features. For example, the near patient access port 72 may include one or more of an offset tubing port vortex-creating feature, a proximal flow-diverting feature, anti-microbial NFC lubricant, anti-microbial eluting surface coating(s) or insert(s), etc.

Near patient access port 72 further includes a high pressure injection port 76 positioned near a distal end thereof. In some embodiments, the high pressure injection port 76 includes a secondary branch 78 that is coupled to the integrated extension set 32, with the integrated extension set 32 further being coupled to a proximal portion 36 at a proximal end thereof. In this way, the near patient access port 72 may receive a high pressure injection via the proximal portion 36 and integrated extension set 32.

In some embodiments, the high pressure injection port 76 is configured as a y-adapter. Additionally and/or alternatively, in some embodiments, high pressure injection port 76 may be color-coded and/or include indicia indicative of maximum flow rate, catheter length, maximum pressure rating, etc.

Furthermore, while not shown in FIG. 5, the catheter of the catheter of system 70 may include a reinforced catheter tip and/or fenestrations in the catheter tip. In this way, the catheter is capable of improved gravity and high-pressure flow rate.

While several embodiments of integrated midline catheter systems having near patient access ports for peripheral probe device access were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. An integrated midline catheter system comprising:
a catheter adapter having a catheter extending therefrom, a stabilization platform, a proximal connector portion fluidly coupled to the catheter, and a side branch fluidly coupled to the catheter;
a near patient access port coupled to the side branch of the catheter adapter, wherein the near patient access port comprises a connector portion couplable to a peripheral probe device; and
a catheter insertion device, wherein the catheter insertion device is configured to enable distal movement of the catheter adapter and catheter so as to position the catheter within a patient's vasculature.

2. The system of claim 1, wherein the near patient access port further comprises a secondary port, and wherein the secondary port is coupled to an integrated extension set.

3. The system of claim 2, further comprising a proximal access port coupled to a proximal end portion of the integrated extension set.

4. The system of claim 3, wherein the proximal access port is color-coded to provide a flow rate indication.

5. The system of claim 3, wherein the proximal access port comprises indicia indicative of at least one of catheter length and catheter gauge.

6. The system of claim 1, wherein the connector portion of the near patient access port is a needle-free connector.

7. The system of claim 6, wherein the needle-free connector is a split septum needle-free connector.

8. The system of claim 1, wherein the near patient access port further comprises a high-pressure injection port.

9. The system of claim 8, wherein the high-pressure injection port is color-coded to provide a flow rate indication.

10. The system of claim 8, wherein the high-pressure injection port includes indicia indicative of at least one of catheter length and catheter gauge.

11. The system of claim 1, wherein the stabilization platform comprises a pair of stabilizing wings.

12. The system of claim 11, wherein each of the stabilizing wings comprises a gripping surface to provide a grip for the distal movement of the catheter adapter and catheter along the catheter insertion device.

13. The system of claim 1, further comprising a pair of catheter advancement tabs extending from the catheter adapter and configured to provide to provide a grip for the distal movement of the catheter adapter and catheter along the catheter insertion device.

14. The system of claim 13, wherein the catheter advancement tabs are detachable from the catheter adapter.

15. The system of claim 1, wherein the catheter insertion device further comprises a housing, an introducer needle extending distally from the housing, and an actuator tab slidable along the housing to selectively advance a guidewire through the introducer needle, wherein the catheter is configured to selectively advance over the introducer needle and the guidewire.

16. The system of claim 1, wherein the catheter insertion device is detachable from the catheter adapter after insertion of the catheter within the patient's vasculature.

17. An integrated midline catheter system comprising:
a catheter adapter having a catheter extending therefrom, a stabilization platform, a proximal connector portion fluidly coupled to the catheter, and a side branch fluidly coupled to the catheter; and
a near patient access port coupled to the side branch of the catheter adapter, wherein the near patient access port comprises a connector portion couplable to a peripheral probe device.

18. The system of claim 17, wherein the catheter is between 8 cm and 10 cm in length.

19. The system of claim 17, wherein the near patient access port further comprises a secondary port, and wherein the secondary port is coupled to an integrated extension set.

20. The system of claim 17, wherein the connector portion of the near patient access port is a needle-free connector.
